(19) United States
(12) Patent Application Publication
(10) Pub. No.: US 2005/0048090 A1
(43) Pub. Date: Mar. 3, 2005

(54) Temperature changing skin care product

(76) Inventor: Allen H. Rau, Cincinnati, OH (US)

Correspondence Address: Supervisor, Patent Prosecution Services;PIPER RUDNICK LLP, 1200 Nineteenth Street, N.W., Washington, DC, 20036-2412, US

(21) Appl. No.: 10/924,822

(22) Filed: Aug. 25, 2004

Related U.S. Application Data
(60) Provisional application No. 60/499,385, filed on Sep. 3, 2003.

(51) Int. Cl. A61F 7/00; A61K 7/00

(52) U.S. Cl. 424/401

(57) ABSTRACT

Novel heat generating compositions, which are suitable for topical application to the skin of a subject are provided. More particularly, topically applied anhydrous or substantially anhydrous compositions containing at least one inorganic or organic salt, which has a positive heat of solution that, upon contact with water, produce a warming effect on the skin of the subject are provided. Also provided is a method of making novel heat generating composition, which includes a method of controlling the change in temperature of the heat generating composition.

This application claims priority from U.S. Provisional Application Ser. No. 60/499,385 filed Sep. 3, 2003. The entirety of that provisional application is incorporated herein by reference.

### BACKGROUND OF THE INVENTION

1. Field of the Invention

The present invention relates generally to novel compositions, which are suitable for topical application to the skin of a subject. More particularly, the present invention relates to topically applied compositions, which are substantially anhydrous or preferably anhydrous, the compositions containing at least one inorganic or organic salt, which has a positive heat of solution that, upon contact with water, produce a warming effect on the skin of the subject. The present invention also relates to a method of making the heat-releasing composition of the present invention. The invention also relates to a method of controlling the change in temperature of a heat-releasing composition that is topically applied to the skin of a subject.

1. Background of the Technology

Topically applied cosmetic or therapeutic skin care products that become warm during use are well known. Such compositions include facial cleansers, skin creams, lotions, soaps, detergents, topical pharmaceuticals, shaving cream and shaving lotion formulations, all of which produce heat by chemical action with water or other protic solvents to provide a heat generating topical composition.

The proliferation of heat generating topically-applied compositions is attributable to the soothing effect of the warmth generated thereby. For example, U.S. Pat. No. 6,309,655 discloses a cosmetic composition which combines a warming effect contributed by zeolite being exposed to water during the skin cleansing process. U.S. Pat. No. 4,379,143 and U.S. Pat. No. 4,626,550 are additional examples of self-warming cosmetic compositions containing anhydrous zeolite that is subsequently exposed to water. The self-warming component zeolite has also been included in compositions formulated for use as toothpastes (U.S. Pat. Nos. 4,349,533; 4,159,316; 4,187,287, and 3,250,680). U.S. Pat. No. 5,757,004 discloses a similar toothpaste composition that employs hydratable salts in low molecular weight solvents that can include propylene glycol, glycol, glycerin. Heat generating agents, such as zeolite have also been used in cleansing and medicated creams (U.S. Pat. No. 4,362,715), analgesic balms (U.S. Pat. No. 4,379,143), and the like. A particular negative effect of using some zeolite materials is that a composition of high alkalinity (as high as pH 12) can result. The sensitive skin of a subject can have only very limited exposure to such compositions (as little as 1-2 minutes is preferred).

Whether the warming composition is applied for purposes of shaving or cosmetic application or for therapeutic uses, such as muscle massage or medication application, the effect is equally positive to the subject, a warming sensation that relaxes the skin and underlying muscles rather than causing a negative response to contact with a cold lotion or composition.

In addition to the commonly used zeolite, other heat-generating ingredients have been employed in a variety of compositions. U.S. Pat. No. 3,341,418 discloses a shaving composition wherein heat is generated by an oxidation-reduction reaction, which occurs when an oxidant, such as hydrogen peroxide or urea hydrogen peroxide from one compartment of the dispensing unit is mixed with reductants from a second compartment, which includes thiourea and a compound such as 1-phenyl-2-thio barbituric acid. U.S. Pat. No. 3,702,302 also employs the heat generation from an oxidation-reduction reaction using potassium sulfate as the reductant component. Similarly, U.S. Pat. No. 4,088,751 discloses a cosmetic composition that also uses specific reductants to effect heat generation from an oxidation-reduction reaction.

U.S. Pat. No. 4,110,426 discloses a heat generating cosmetic composition in a surfactant carrier formulation, wherein compounds having at least one boron-oxygen-born linkage react exothermically with water or other protic materials to release heat.

U.S. Pat. No. 6,540,989 discloses a heat generating rinse-out hair conditioner that relies on glycol as the warming agent. The warming effect is achieved when the glycol comes into contact with water.

Each of the above described conventionally used self-warming products are typically anhydrous formulations that release heat when they come in contact with water. The component material used to generate heat in such compositions is conventionally a poly-hydroxy compound (such as glycerin or polyethylene glycol) or zeolite. The amount of heat that these materials generate is noticeable to the user, but the level of heat generated is not very intense and can result in user dissatisfaction. Further, such conventional compositions do not provide a predictable means of varying the rate or the level of heat released during use.

There remains therefore a need to provide a topically applied composition that can be used in a wide variety of skin care applications with the added benefit of generating heat after being applied to the skin, if need be at higher temperatures and for longer time periods than are available from conventionally used self-warming topically applied products. Further, there remains a need for such a composition, wherein the rate of heat released and the amount of heat released can be controlled by pre-selecting specific components for the composition produced.

### SUMMARY OF THE INVENTION

The present invention provides a topically applied, substantially anhydrous, composition containing at least one inorganic or organic salt, the salt having a specific heat of solution value, that upon contact with water effects a change in temperature of the composition based upon the value of the heat of solution of the salt, the composition being selectively formulated for a variety of skin care applications.

Another object of the present invention is a topically applied, substantially anhydrous, composition containing at least one inorganic or organic salt having a positive heat of solution, the composition being selectively formulated for a variety of skin care applications, wherein the warming effect is the result of contacting the composition to water.

Another object of the invention is to provide a substantially anhydrous, topically applied skin care composition having a selected heat generating ingredient, which upon contact with water can release a predetermined level of heat for a predetermined period of time.

Another object of the invention is to provide a substantially anhydrous, topically applied, self-warming composition that is formulated for use as a skin cleanser.

Another object of the invention is to provide a substantially anhydrous, topically applied, self-warming composition that is formulated as a carrier composition for therapeutic agents, which when the composition is applied to the skin of as subject, it facilitates the delivery of the therapeutic agent.

Another object of the invention is to provide a substantially anhydrous composition that can be employed with patch-type agent delivery systems that, upon application of the patch to the skin and moisture contact with the composition, releases heat that warms the area of the skin contacted by the patch and the included agent thus facilitating uptake of the agent into the skin due to increased blood flow to the area.

Another object of the invention is to provide a method of making a substantially anhydrous composition having a heat generating component.

### DETAILED DESCRIPTION OF THE INVENTION

The following discussion and accompanying examples of representative heat generating compositions describe the present invention, which is limited only by the claims attached hereto.

Various substances are known to have different heats of solution, some being exothermic and others being endothermic. Examples of such compounds are listed in Lange's Handbook of Chemistry, 11^{th} edition, Table 9-6. The greater the value of the heat of solution, the more heat is liberated per gram mole of the substance. Thus, materials with higher heats of solution are able to raise the temperature of a given amount of water higher than compounds with lower heats of solution.

Examples of materials with relatively high heats of solution (Kilo-calories per gram mole of the compound) are magnesium chloride (+35.92), calcium chloride (+17.41), calcium oxide (+18.33), barium oxide (+11.40), magnesium sulfate (+20.28), ferric chloride (+32.68), ferrous chloride (+5.65), aluminum sulfate hexahydrate (+8.02), and aluminum chloride (+76.3).

Generally, compositions of the present invention, depending upon the level of heat to be generated during use, will be formulated to have from about 1 to 50 wt. % heat generating compound of the total composition. Preferably the composition will have between 10 to 50 wt. % and more preferably between 20 to 40 wt. % of heat generating compound. The amount of heat typically generated by the present composition can be higher than the temperatures normally produced by the use of the conventionally used zeolite and should be determined by the specific need of the application and the comfort level of the subject. It is within the concept of the invention to provide formulations, which upon activation produce temperatures that can easily range from 25 to 50° C., preferably 30 to 45° C., and more preferably 37 to 45° C. Temperatures generated by compositions of the present invention for many topical-use applications can be 40° C. or higher.

Compositions of the present invention can be selectively formulated to provide different levels of heat upon activation as well as to provide differing periods of time over which the heat is released from the composition. For compositions requiring higher levels of heat generation, an inorganic or organic salt having a high heat of solution can be selected as the heat generating ingredient. In contrast, exothermic salts, which have lower heats of solution can be selected for compositions that are designed to release lower levels of heat upon contact with water.

It is also within the concept of the present invention to include more than one heat generating component, a portion of which can be coated or encapsulated with a time-release or mechanically-released protective coating such as is commonly used to protect active agents from immediate contact with the surrounding environment. Formulations of the present invention thus treated would allow un-encapsulated, unprotected molecules to react to the presence of water and generate heat immediately, while those heat generating compounds that were encapsulated would be at least temporarily protected from contact with water and thus would not generate heat until the protecting encapsulation layer was dissolved or degraded by the environment. Any encapsulation method or material known in the field of providing time-released active agents can be employed with the present invention providing that the encapsulation process material does not itself activate the heat generating agent and thus cause the premature release of heat therefrom.

The physical characteristics of the particles of heat generating agent can also be varied so as to effect the amount of surface area of the particle that is exposed to the moisture present at the time of use. Larger sized particles would present a smaller total surface area for the heat generating compound exposed to any water present and would therefore react with the water more slowly with lower levels of heat being released over a longer period of time.

The rate of heat generation by the composition can also be controlled by selection of the type of carrier used for the composition. Preferably, a hydrophobic carrier is selected to avoid premature activation of the heat generating component. When using a hydrophobic carrier, it is possible to include a surfactant ingredient to increase the water compatibility of the carrier and thus increase the rate of heat released by the composition.

The heat generating component can be incorporated into cosmetically acceptable anhydrous base product formulations. A cosmetically acceptable component or formulation is one that is not harmful or irritating to the skin to which it is applied over a sustained period of time, such as 24 hours. The formulations for every possible application of the present invention must be substantially anhydrous, that is have less than 3% water content, and preferably are anhydrous, less than 1% water content, since any water present can result in dissolution of the heat generating active ingredient, the inorganic or organic salt. Early activation of the exothermic salt would prematurely release the heat of solution for that salt and thus diminish the warming effect of the composition when applied to the skin.

The composition can be formulated as a dry powder or liquid formulation so long as the composition remains substantially anhydrous until just prior to or at the same time as the application of the composition to the subject's skin.

A suitable carrier for use in dry powder compositions of the present invention can include natural polymers such as corn starch, modified corn starch, pre-gellatinized corn starch, talc, tapioca starch, and the like. Other finely divided solids such as clay, microcrystalline cellulose, silca, alumina, and the like can also be used as solid carriers in the composition. Other polymers can also be included in such a dry power formulation, such as for example polyethylene glycol powder, preferably of higher molecular weights over 1500 MW, such as 4000 to 8000 MW and more preferably from 6000 to 8000 MW.

Liquid formulations of the heat generating composition can include preferred carriers such as oils. Suitable carriers include, for example mineral oils, vegetable oils, hydrocarbon oils, silicones, silicone waxes, fatty acid esters, paraffins. Preferably mineral oils, silicone oils, and plant oils can be used. Prepared polymeric carrier media can also be employed so long as such carriers promote the anhydrous character of the composition. Thus, the heat generating composition can be incorporated into any non-aqueous inert carrier or extending medium which is non-reactive with the composition.

Additional suitable inert carriers or extending media include particulate solids, such as polyethylene and other organic polymers as well as aluminum oxide and other inert inorganic materials, inert solvents such as straight chain, branched, cyclic saturated and unsaturated hydrocarbons such as benzene, toluene, mineral oil, additional botanical oils.

Buffers, which are well known for use in topical applications can be included in the composition to maintain a pH at the skin surface in the range of about 4.5 to 9, preferably in the range of about 5.5 to 8.5, more preferably in the range of about 5.5 to 7.5 during the period of application and wear of the composition. Stabilizing the pH of the composition by the use of such buffers can enhance the experience of using the composition and minimize the possibility of damage to the skin.

Other additives that can be included in the composition are emulsifying agents and thickening or bodying agents, which may be added as needed to effect the desired consistency. Additionally perfumes, emollients, foaming agents, colorants, preservatives, and other additives conventionally used in topically applied products may also be added as desired on the condition that such additional ingredients do not cause or promote early hydration of the composition and the resultant premature activation of the heat generating ingredient. Preferably, additional additives can be included in the amounts necessary to support the specific purpose of that formulation but will not be included in amounts so great as to interfere with the substantially anhydrous nature of the composition.

Any topically applied product can be formulated to include the heat generating composition disclosed herein. Non-limiting examples of such products include cosmetics such as skin treatments, cleansers, plasters, pads, lotions, creams, gels, ointments, solutions, shampoos, tanning products, lipsticks, and the like.

Therapeutic ingredients can also be included in the heat generating composition to include, for example acne treatments, fungal treatments, antimicrobial treatments, analgesic applications, topical hormonal treatments, anti-smoking nicotine treatments and appetite suppressants. Such topical applications can be direct, that is applied directly to the skin; or indirect, that is applied first to a carrier such as patch or bandage, which is subsequently brought into contact with the skin.

In addition, a skin-warming composition can be beneficial in prompting increased blood flow close to the skin and thus promote passage of skin absorbed medicament additives into the blood supply. This is particularly true for skin medicaments such as those used to treat acne or other persistent skin disorders. This warming effect on the skin can be of particular benefit in the wide-range of applications of therapeutic and medicated patches that have proliferated in recent years. For example, U.S. Pat. No. 6,652,876 discloses the use of a patch applied to the skin for the purpose of delivery of nonsteroidal antirheumatics and U.S. Pat. No. 6,719,997 discloses the use of patch technology for the transdermal delivery of active amines. In the field of immunization, for example, U.S. Pat. No. 5,980,898, discloses a patch application technology for transdermally transporting an antigen, which elicits an immune response through intact skin. Such antigen transport and pick-up by the subject, as with the other patch technology applications may be facilitated by an increased blood flow to the area of the skin where the patch is applied if a composition can be applied to the area that will locally increase the skin warmth and blood flow. The commonly used heat-generating agent zeolite provides a generally low level of heat that is often considered inadequate for the more intense requirements of therapeutic applications to the skin. Further, as indicated above, some zeolite containing compositions tend to be too alkaline and therefore caustic to the skin thus restricting the amount of time that such compositions can be left in place on the skin.

Importantly, the carrier media selected for formulations of the heat generating composition as well as additional active or supporting ingredients must all be of a nature that does not interfere with or prematurely degrade the anhydrous nature of the inventive composition. In addition to the anhydrous carrier, the composition can include as necessary an additional active ingredient, emusifiers, surfactants, detergents, foaming agents, wetting agents, thickeners, film forming agents, beneficial agents such as vitamins, and other additives that are preferably used in skin treatment formulations.

Among the numerous anhydrous carrier media that might be suitable for the composition, Sunflower oil or a Sunflower oil mixture including thickeners, abrasives, and vitamins (such as Vitamin E) are preferred. Alternatively, other anhydrous carrier media can be used provided such carrier media are supportive of the anhydrous characteristic of the heat generating composition and are cosmetically acceptable.

Formulations including the heat generating composition can, optionally include a skin conditioning agent. The term skin conditioning agents as used herein means a material which provides a skin conditioning benefit. For example, the skin conditioning agents provide a therapeutic or cosmetic benefit to the skin including, but not limited to, moisturization, emolliency, visual improvement of the skin surface, soothing of the skin, and softening of the skin. Suitable skin conditioning agents for use in the present invention include dimethicone, dimethicone copolyol, petrolatum, hydrogenated polyisobutene, mineral oil, esters such as isopropyl myristate, isopropyl palmitate, butyl stearate, hexyl laurate, and the like.), and additional oils beyond that employed as the formulation carrier, such as cotton seed oil, caster oil, corn oil, and the like.

An additional natural polymer that can be, and for some formulations, preferably is included is pregelatinized starch. Pregelantinized (pregelled) starch is obtained from botanicals by processing the starch either chemically or mechanically, by processes well-known in the art so as to rupture all or part of the granules in the presence of water. The product is then dried and can be used in cosmetic compositions and skin lotions. Such pregelled starches are commercially produced from botanical sources such as, corn, tapioca, potato, wheat, rice, and the like. In the present invention, pregelled corn starch is the preferred composition component.

Another often preferred material for formulations of the heat generating composition is modified corn starch. Modified corn starch is a often used thickening ingredient that is non-reactive with the anhydrous heat generating ingredient of the composition. Corn starch has two components: amylose (a straight chain polymer of glucose) and amylopectin (a branched chain polymer of glucose). Commonly corn starch is modified chemically (with a variety of approved agents) or physically to improve the consistency of the material for use as a thickener.

Also included in some formulations of the composition is a stabilizing polymer, such as polyethylene glycol (PEG). Polyethylene glycol is a polymer produced in a wide range of molecular weights, the value of which can greatly effect the characteristics of the polymer. Polyethylene glycols of higher molecular weight have a higher flash melting point and tend to be less sticky and are solid at room temperature. Lower molecular weight PEG can be liquid at ambient temperature and tends to react with the heat generating component causing premature heat release. PEG with a melting point higher than ambient temperature is preferred. For this reason, higher molecular weight PEG is preferred, as in the examples provided below. PEG in excess of 1500 MW is preferred, 2000 MW or higher is more preferred; 4000 to 8000 MW is still more preferred and 6000 to 8000 MW is most preferred.

Additionally, silicone oils such as cyclomethicone can be included in the composition to stabilize the consistence of the lotion or oil. Cyclomethicone is particularly preferred as it is clear, colorless, and odorless and has been commonly used for prolonged periods on the skin without adverse effect.

Emulsifiers can also be included in the composition of the present invention to stabilize the components in a consistent formulation. Any known emulsifier that is cosmetically acceptable and will not interfere with the anhydrous nature of the composition can be employed in formulations of the present invention. While the following non-limiting examples include the emulsifier polysorbate 80, it remains that any cosmetically acceptable emulsifier can be acceptable.

Additional thickeners and fillers that will support a stable emulsion including the anhydrous composition can also be used. A preferred natural thickener is cosmetically acceptable clay, such as Kaolin. Kaolin is a clay composed essentially of the mineral kaolinite. While Kaolin has many industrial uses, it has been found to be particularly well-suited for use in cosmetic applications; such as mud masks. Such a formulation is included in the examples below.

### EXAMPLES

The following non-limiting examples are further illustrative of the varied formulations of the present invention.

An example formulation of a base product, which would provide an acceptable anhydrous media in which to include a heat generating compound of the present invention is provided below in Table 1. Depending upon the amount of heat generating compound added to the base product formulation, the amount of carrier component as well as other ingredients would be adjusted.

TABLE 1
Base Product

| Component | % w/w |
|---|---|
| Sunflower Oil, Soybean Germ Extract | 75.0 |
| Silica, Corn Starch and Tocopherol* | |
| (Natunola ® Sunflower 1102)** | |
| Cyclomethicone | 2.5 |
| Corn Starch Modified | 15.0 |
| Emulsifiers | 2.5 |
| Fragrance, Preservative | 5.0 |

*Tocopherol - Vitamin E
**Natunola Health; Ottawa, Ontario, Canada

As indicated above, anhydrous powder, granular base products, and specific use liquid formulations, which incorporate materials with relatively high heats of solution, can be formulated. The base product formulation shown above in Table 1 can be modified with one or more heat generating compounds having relatively high heats of solution and formulated to meet the particular needs of specialized products, such as, for example, a warming cream formulation as shown in Table 2, a warming mud mask formulation as shown in Table 3, a powdered mud mask as shown in Table 4, a warming facial cleanser powder as shown in Table 5, and a warming cleansing cream as shown in Table 6. The example products shown in the tables serve to demonstrate how the heat generating composition can be formulated to meet a variety of uses. Other non-limiting examples include analgesic rubs, depilatory creams, medicated creams, lotions or salves, etc. Each of the above formulations are provided as non-limiting examples of the basic formulations that can be adapted for different specific uses of the product of the present invention.

TABLE 2
Warming Cream

| Component | % w/w |
|---|---|
| Sunflower Oil, Soybean Germ Extract | 53.7 |
| Silica, Corn Starch and Tocopherol* | |
| (Natunola ® Sunflower 1102)** | |
| Magnesium Chloride (anhydrous) | 30.0 |
| Cyclomethicone | 2.0 |
| Corn Starch Modified | 10.0 |
| PEG-40 Sorbitan Peroleate | 1.0 |
| Polysorbate 80 | 1.0 |
| Preservative | 0.3 |
| Fragrance | 5.0 |

*Tocopherol - Vitamin E
**Natunola Health; Ottawa, Ontario, Canada

TABLE 3
Warming Mud Mask

| Component | % w/w |
|---|---|
| Sunflower Oil | 24.0 |
| Magnesium Chloride (anhydrous) | 30.0 |
| Kaolin | 40.0 |
| PEG-40 Sorbitan Peroleate | 2.0 |
| Polysorbate 80 | 2.0 |
| Preservative | 0.3 |
| Fragrance | 1.7 |

TABLE 4
Powdered Mud Mask

| Component | % w/w |
|---|---|
| Magnesium Chloride (anhydrous) | 30.0 |
| Kaolin | 45.0 |
| Pregelled Corn Starch | 20.0 |
| Fragrance | 5.0 |

TABLE 5
Warming Facial Cleanser Powder

| Component | % w/w |
|---|---|
| Magnesium Chloride (anhydrous) | 20.0 |
| Sodium Lauryl Sulfoacetate | 15.0 |
| Corn Starch | 59.5 |
| Polyethylene Gylcol Powder | 4.0 |
| (MW 8000) | |
| Fragrance | 1.5 |

TABLE 6
Warming Cleansing Cream

| Component | % w/w |
|---|---|
| Sunflower Oil, Soybean Germ Extract | 52.7 |
| Silica, Corn Starch and Tocopherol* | |
| (Natunola ® Sunflower 1102)** | |
| Magnesium Chloride (anhydrous) | 30.0 |
| Sodium Lauryl Sulfoacetate | 10.0 |
| PEG-40 Sorbitan Peroleate | 2.5 |
| Polysorbate 80 | 2.5 |
| Preservative | 0.3 |
| Fragrance | 2.0 |

*Tocopherol - Vitamin E
**Natunola Health; Ottawa, Ontario, Canada

The heat generating exothermic salt of the composition can be incorporated into the anhydrous base at any amount that results in a product that provides the desired level of heat and is aesthetically acceptable for its intended use. The amounts of exothermic salt that can be included in a typical composition can range from 10 wt. % to 80 wt. % and preferably from 20 wt. % to 70 wt. % and more preferably between 20 to 60 wt. % depending upon the exothermic salt selected, the purpose and degree of heat required from the composition, and the nature of the carrier media to be used in the product.

Since the exothermic material will generally be a solid, its particle size can be chosen to be appropriate for the intended use of the product. The range of particle size will depend on the particular salt selected and the particular use of the final product. For instance, for cleansing products a larger, slightly coarse particle size might be selected over a smaller, more gentle feel particle, which would be used in a cream composition. For an exfoliating product, it may be desirable to use a fairly coarse particle size. Alternately, a delicate skin lotion may require a very fine particle size. As indicated earlier, it should be noted that the particle size will be inversely proportional to the total surface area of the heat generating particulate ingredient and thus may also affect its dissolution rate, thus enabling the formulator to have control over the rate of heat release from the composition.

Cleansing formulations, which contain the heat-generating active ingredient, can also include soaps, detergents, and other cleaning compositions so long as the cleansing ingredient is supportive of the anhydrous nature of the composition and is suitable for use on the skin.

The composition can include a surface active agent of the non-ionic type such as an ethylene oxide condensation formulation with higher fatty alcohols, higher fatty acids, higher fatty acid amides, alkylated phenols such as nonyl phenols and dinonyl phenols. Other non-ionic surfactants suitable for use with the heat-generating active ingredient are the polypropylene glycols, amide and amine condensates such as fatty acid diethanol-amide (lauric and myristic diethanolamide). Other organic surfactants can be utilized with the heat-generating ingredient in the form of an emulsion, dispersion, or suspension.

Among the anionic surfactants that may be used are the aliphatic sulfated or sulfonated detergents. Suitable examples of these aliphatic detergents are sulfuric acid esters of polyhydric alcohols incompletely esterified with higher fatty acids, either saturated or unsaturated, such as, for example, coconut oil monoglycerride monosulfate lauroyl monoglyceride monosulfate; the long chain pure or mixed higher alkyl sulfates, such as lauryl sulfate, cetyl sulfate, higher fatty alcohol sulfates derived from hydrogenated or non-hydrogenated coconut oil or tallow fatty acids; the higher fatty acid esters of hydroxy alkyl sulfonic acids; higher fatty acid amides of amino alkyl sulfonic acids, such as the oleic acid amide of amino methyl sulfonic acid, the lauric acid amide of taurine, and the like.

Other acceptable aliphatic sulfonates include fatty sulfoacetates, such as coconut fatty alcohol sulfoacetates; sulfated fatty acyl monoethanolamides, such as sulfated lauroyl monoethanolamide; fatty sulfoacetamides, such as lauryl sulfoacetamide; lower alkyl sulfoccucineates, such as dioctyl sulfossucinate; and sulfonated fatty oils such as dictyl sulfosuccinate; sulfonated fatty oils such as sulfonated ester oil and sulfonated red oil, and lower alkyl esters of alphsulfonated higher fatty acids, such as methyl ester of alphasulfo mystric acid, sodium salt, and the alkyl sulfonates.

Synthetic detergents having a carboxylate group, and particularly the higher fatty acid amides of aliphatic amino acid compounds may also be included. Suitable detergents include higher fatty acyl sarcosinates such as water-soluble salts of N-lauroyl or N-cocoyl sarcosine. Other suitable materials are the higher fatty acid amides of polypeptide amino acids obtained by protein hydrolysis, detergents with carboxylate groups, and ether-containing sulfates such as lauryl phenol polyethylenoxy sulfates, and alkyl polyglycol ether sulfates.

Other anionic detergents which may be employed also include water-soluble alkyl phosphates and soaps such as the sodium, potassium and triethanolamine soaps of fatty acids. Examples are sodium laurate, sodium palmitate, sodium ooleate and the potassium and/or triethanolamine soaps of coconut oil, palm oil, and tallow fatty acids. Other suitable detergents are alkyl sulfates, olefin sulfonates and alkyl phosphonates.

Cationic detergents wherein a quaternary nitrogen is part of an open chain or heterocyclic structure may also be used alone or in combination with other compatible detergents. Suitable salts are the chloride, bromide, acetate, sulfate, and methosulfate, and the like. Examples are lauroyl pyridinium bromide, N(lauroyl colamino formylmethyl) pyridnium chloride, cetyl trimethyl ammonium chloride, cetyl pyridinium chloride, stearyl or oleyl dimethlbenzyl ammonium chloride, distearyl dimethly ammonium salts, stearyl amine acetate, stearyl dimethly ammonium salts, stearyl amine acetate, stearryl dimethyl amino hydrochloride. Other suitable detergents are imidazolinium quaternary compounds and phosphorium salts.

Other suitable surface-active agents which can have a cationic nature and which may be use in compositions containing the heat-generating ingredient of the present invention include the higher alkyl amine oxides such as lauryl dimethly amine oxide. Other long chain alkyl radicals can be used in place of the lauryl radical.

Any of the conventionally used amphoteric detergent materials may be employed in the present composition. Among those are fatty or higher alkyl imidazolines, and higher alkyl betaalanines.

Detergents having an intermediate linkage between the hydrophobic aliphatic hydrocarbon group and the water-solubilizing sulfate, sulfonate or carboxylate group are acceptable components since such materials are considered to be relatively mild and non-irritating to the skin. Such intermediate linkages are amide, ether, polyether, ester and amine groups as illustrated by the sarcosinates, imidazolines and like materials.

The self-heating cosmetic composition of the present invention may be prepared in a variety of physical forms. Formulations may be liquids, pastes, solids, and powders. The formulations may constitute a single phase or may comprise an emulsion of two non-aqueous phases made miscible by agitation (i.e., mineral oil or botanical oils and a polymeric suspension). The formulations can be in the form of a dispersion of solid components in a non-aqueous liquid phase. It is also within the concept of the invention that the formulations of the heat generating composition may exist in the form of a dual phase (aqueous activator and the anhydrous composition of the present invention that are contained separately and admixed only at the site of application.

The resulting products of the heat generating composition can be applied directly to the skin, the skin being wetted by use of a water spray, splash, or submersion before, after or during application of the composition. Alternatively, the substantially anhydrous or anhydrous composition can be admixed with the activating water containing component immediately prior to application of the activated composition to the skin. It is within the concept of the invention to effect the admixing of the composition with the water activator simultaneously with the application to the skin by use of separate application devices (syringes, bottles, tubes, etc.) or by admixing the composition and water activator in an specially designed container/applicator having a mixing antechamber just before the opening nozzle from which the activated composition is applied to the skin. In every case, when the water or water containing activator is mixed with the heat generating composition, noticeable heat is released.

It is also within the scope of the present invention that the anhydrous composition containing the active heat-generating ingredient can be applied to the skin of a subject and the naturally occurring moisture from the skin and surrounding atmosphere can slowly activate the composition to generate the desired warming effect. Such naturally produced activating moisture can also be employed to activate applications of the present invention used with patch technologies as discussed above.

An effective exothermic material component can have a minimum heat of solution of about 2-15 kcal/gram mole, preferably 5-10 kcal/gram mole and most preferably 7-10 kcal/gram mole in order to deliver the intense heating effect on the skin that is anticipated by this invention.

The composition can be formulated such that the heat released per gram of inventive products will be generated quickly and in excess of that delivered by products that use glycerin, polyethylene glycol or zeolite to provide self heating effects.

The concept of the invention can include the use of materials characterized by endothermic heats of solution, which when included in similar compositions can provide a cooling effect to the skin to which the compositions are topically applied. Some examples of such endothermic heat adsorbing materials as found in Lange 's Handbook of Chemistry, 11^{th} edition, Table 9-6. The lesser the value of the heat of solution, the more potential for absorption of heat and thus the more cooling effect would be experienced. Examples of such compounds are ammonium chloride, ammonium nitrate, barium oxide, ferric nitrate, magnesium sulfate heptahydrate, potassium nitrate, sodium tetraborate decahydrate, sodium nitrate, and sodium sulfate decahydrate. Products that can be produced using such endothermic compounds could be used as a soothing lotion to ease the discomfort of a subject. For example, a cooling lotion could be used to sooth the effect of high ambient temperatures experienced by a subject. Such endothermic composition products could be used alone or as a carrier of an analgesic ingredient that could sooth a subject's sensation of pain resulting from burns, including sunburn or pain experienced due to tired or injured muscles after physical exertion.

For such endothermic applications, the formulations of the various specific use products can be designed very similarly to the examples presented above for exothermic, heat generating compositions, with the distinct difference being the cooling sensation for these latter described products.

All references cited with this application are herein fully incorporated by reference. Variations, modifications, and additions to this invention will be readily apparent to one skilled in the art and such modifications and additions would be fully within the scope of the invention, which is limited only by the following claims.

1. A topically applied, anhydrous skin treatment composition comprising:
an anhydrous carrier component, said carrier component being cosmetically acceptable;
a salt component, said salt component having a specific heat of solution that when said salt is contacted with water effects a temperature change in said composition.

2. A skin treatment composition according to claim 1, wherein said salt component is an exothermic salt having a positive heat of solution.

3. A skin treatment composition according to claim 1, wherein said carrier component is anhydrous.

4. A skin treatment composition according to claim 3, wherein said carrier component is selected from the group consisting of mineral oils, vegetable oils, hydrocarbon oils, silicones, silicone waxes, fatty acid esters, and paraffins.

5. A skin treatment composition according to claim 4, further comprising at least one additional component selected from the group consisting of modified corn starch, pregelatinized corn starch, and Kaolin.

6. A skin treatment composition according to claim 4, wherein said salt is an exothermic inorganic or organic salt.

7. A skin treatment composition according to claim 6, wherein said salt is an inorganic salt selected from the group consisting of magnesium chloride, calcium chloride, calcium oxide, barium oxide, magnesium sulfate, ferric chloride, ferrous chloride, aluminum sulfate hexahydrate, and aluminum chloride.

8. A skin treatment composition according to claim 1, further comprising an emulsifier.

9. A skin treatment composition according to claim 1, further comprising a polymeric component.

10. A skin treatment composition according to claim 9, wherein said polymeric component is a polyethylene glycol having a melting point above ambient temperature.

11. A skin treatment composition according to claim 9, wherein said polymeric component is a polyethylene glycol having a molecular weight greater than 2000.

12. A skin treatment composition according to claim 9, wherein said polymeric component is a polyethylene glycol having a molecular weight greater than 6000.

13. A skin treatment composition according to claim 1, wherein said salt is an endothermic salt having a negative heat of solution.

14. A skin treatment composition according to claim 1, wherein said composition is formulated as skin lotion or cream that is a member of the group consisting of a skin tanning cream, skin cleansing cream, skin medicated cream, skin shaving cream, skin lotion, skin gel, skin ointment, skin analgesic ointment, skin depilatory cream, and lipstick.

15. A patch configured to be applied to the skin of a subject comprising:
a dressing, said dressing configured to cause hydration of intact skin;
an anhydrous composition comprising an anhydrous carrier component, said carrier component being cosmetically acceptable and a salt component, said salt component having a specific heat of solution that when said salt is contacted with water effects a temperature change in said composition.

16. A patch according to claim 15, wherein said salt is an anhydrous exothermic salt having a positive heat of solution.

17. A patch according to claim 16, wherein said composition further comprises at least one active ingredient.

18. A patch according to claim 17, wherein said at least one active ingredient is selected from the group consisting of an immunizing antigen, a nicotine-based compound, a hormonal-based compound, an analgesic, an acne medication, a fungal control compound, an antimicrobial compound, and an appetite suppressant compound.

19. A method of producing a topically applied, anhydrous skin treatment composition, the method comprising:
providing an anhydrous carrier component, said carrier component being cosmetically acceptable;
providing a salt component, said salt component having a specific heat of solution that when said salt is contacted with water effects a temperature change in said composition;
combining said carrier component with said salt component to form an anhydrous skin treatment composition.
